(19) United States
(12) Patent Application Publication
(10) Pub. No.: US 2010/0279322 A1
(43) Pub. Date: Nov. 4, 2010

(54) Direct detection of intracellular fluorescently tagged cells in solution

(75) Inventors: Cha-Mei Tang, Potomac, MD (US); Peixuan Zhu, Derwood, MD (US); Daniel L. Adams, Kensington, MD (US)

Correspondence Address: ROYLANCE, ABRAMS, BERDO & GOODMAN, L.L.P., 1300 19TH STREET, N.W., SUITE 600, WASHINGTON,, DC, 20036, US

(73) Assignee: Creatv MicroTech, Inc., Potomac, MD (US)

(21) Appl. No.: 12/773,370

(22) Filed: May 4, 2010

Related U.S. Application Data
(60) Provisional application No. 61/175,273, filed on May 4, 2009.

(51) Int. Cl. C12Q 1/06; G01N 33/552; G01N 33/574; G01N 33/53; G01N 33/569; G01N 33/551

(52) U.S. Cl. 435/7.23; 435/39; 435/7.2; 435/7.21; 435/7.37; 435/7.1

(57) ABSTRACT

A method of detecting target cells and pathogens in a test sample concentrates to target cells in solution by filtering or capturing the target cells on a solid support. The target cells are tagged with a fluorescent dye and dispersed in a solution or suspension. The resulting solution or suspension are introduced to a fluorometer to specifically identify and quantitate the target cells. The target cells can be lysed or whole when introduced to the fluorometer.

### CROSS-REFERENCE TO RELATED APPLICATION

This application claims the benefit under 35 U.S.C. §119(e) of provisional application Ser. No. 61/175,273, filed May 4, 2009, which is hereby incorporated by reference in its entirety.

### FIELD OF THE INVENTION

The invention relates to methods and reagents for the detection of cells from organisms by fluorescent in situ hybridization (FISH) cells or peptide nucleic acid fluorescent in situ hybridization (PNA FISH) in solution using a sensitive fluorometer, not utilizing glass slides or flow cytometry. The invention also describes methods and reagents for detection of intracellular fluorescent tagged proteins, disease markers, etc., in the cells. The invention provides rapid, sensitive and easy to perform assays to identify, characterize, and quantify cells in a solution using a fluorometer without the conventional analysis of images on glass slides. The method of the invention can be applied to the detection of bacteria, yeasts, micro-organisms, and cells from tissue and body fluids, such as tumor cells, fetal cells, epithelial cells, blood cells, and the like. Body fluids can be blood, spinal fluid, saliva, urine, tears, mucus, etc.

### DESCRIPTION OF RELATED ART

Fluorescent In Situ Hybridization (FISH) and peptide nucleic acid (PNA) FISH. The use of FISH has been reported for detection of bacteria directly from blood cultures on glass slides. [Volkhard A. J. Kempf, Karlheinz Trebesius, and Ingo B. Autenrieth, Fluorescent In Situ Hybridization Allows Rapid Identification of Microorganisms in Blood Cultures, Journal Of Clinical Microbiology, 2000, Vol. 38, No. 2, p. 830-838]. The use of PNA FISH probes provides stronger affinities and more rapid kinetics and is now commercially available as kits from AdvanDx (Woburn, Mass.). PNA FISH has shown excellent sensitivity and specificity for detection of pathogens, and can be performed on glass slides within a few hours after bacterial culture. FISH and PNA FISH typically require culturing the sample cells for as much as 8 hours to obtain a volume of cells sufficient for detection. This method also requires the cultured sample to be applied to glass slides and dried before testing. [Kenneth Oliveira, Stephen M. Brecher, Annette Durbin, Daniel S. Shapiro, Donald R. Schwartz, Paola C. De Girolami, Joanna Dakos, Gary W. Procop, Deborah Wilson, Chad S. Hanna, Gerhard Haase, Heidrun Peltroche-Llacsahuanga, Kimberle C. Chapin, Michael C. Musgnug, Michael H. Levi, Cynthia Shoemaker, and Henrik Stender, Direct Identification of Staphylococcus aureus from Positive Culture Bottles, Journal Of Clinical Microbiology, 2003, Vol. 41, No. 2, P. 889-891; Hanna Hartmann, Henrik Stender, Andrea Schafer, Ingo B. Autenrieth, and Volkhard A. J. Kempf, Rapid Identification of Staphylococcus aureus in Blood Cultures by a Combination of Fluorescence In Situ Hybridization Using Peptide Nucleic Acid Probes and Flow Cytometry, Journal Of Clinical Microbiology, 2005, Vol. 43, No. 9 p. 4855-4857; Donna M Hensley, Rachel Tapia, Yadira Encina, An Evaluation of the AdvanDx Staphylococcus aureus/CNS PNA FISH™ Assay Clinical Laboratory Science, 2009, Vol. 22, No. 1, pp. 30-33.]

Recent studies suggest that the use of the S. aureus PNA-FISH assay in the clinical setting can differentiate coagulase-negative staphylococci (CoNS) from S. aureus. The resultant appropriate treatment decreased hospital length of stay for patients with CoNS bacteraemia and the cost associated with antimicrobial treatment [Kenneth Oliveira, Stephen M. Brecher, Annette Durbin, Daniel S. Shapiro, Donald R. Schwartz, Paola C. De Girolami, Joanna Dakos, Gary W. Procop, Deborah Wilson, Chad S. Hanna, Gerhard Haase, Heidrun Peltroche-Llacsahuanga, Kimberle C. Chapin, Michael C. Musgnug, Michael H. Levi, Cynthia Shoemaker, and Henrik Stender, Direct Identification of Staphylococcus aureus from Positive Culture Bottles, Journal Of Clinical Microbiology, 2003, Vol. 41, No. 2, P. 889-891; Forrest et al. 2006].

There is also report of detecting S. aureus cells treated with PNA FISH by flow cytometry. [Hanna Hartmann, Henrik Stender, Andrea Schafer, Ingo B. Autenrieth, and Volkhard A. J. Kempf, Rapid Identification of Staphylococcus aureus in Blood Cultures by a Combination of Fluorescence In Situ Hybridization Using Peptide Nucleic Acid Probes and Flow Cytometry, Journal Of Clinical Microbiology, 2005, Vol. 43, No. 9 p. 4855-4857]

There are several disadvantages of the aforementioned assays: a long blood culture time before the assay itself can begin, sensitivity limited by the use of only one drop of blood culture sample, human interpretation of the microscope slides, and a lack of quantitation.

Disease cells can also be differentiated by fluorescent tag in situ and detected on glass slide or by flow cytometry. One example is a prognostic test for chronic lymphocytic leukemia (CLL) by tagging a marker protein ZAP-70 with fluorescent antibodies in situ of the B cells and detecting the tagged cells by flow cytometry.

### SUMMARY OF THE INVENTION

This invention is directed to methods to provide rapid, sensitive and easy to perform intracellular fluorescent assay in solution (IFAIS) to identify and quantify cells using a fluorometer. This invention can be applied to FISH and PNA FISH detection of cell DNA or RNA in solution. This invention can also be applied to the detection of intracellular proteins in situ of the cells. The methods do not require the tagged cells to be detected on a glass slide or by flow cytometry and, thus, do not rely on a specialist to interpret images on a glass slide. These methods can provide quantitative or semi-quantitative results without the need to culture the cells for extended periods of time. The use of a sensitive fluorometer enables the detection of a low concentration or small number of cells. The method of the invention is able to efficiently and specifically gather the target cells, label the target cells with a fluorescent dye in solution and introduce the solution to a fluorometer to obtain rapid and efficient detection using small samples

The cells may be bacteria, yeast, pathogens, micro-organisms, cells from tissue and body fluids, such as tumor cells, fetal cells, epithelial cells, blood cells, etc. Body fluids may be peripheral blood, spinal fluid, saliva, urine, tears, mucus, etc.

The targets of FISH and PNA FISH in the cells are typically bacterial ribosomal RNA (rRNA) for bacteria, but it can also be DNA or mRNA for identification for other types of cells. The intracellular fluorescence detection concept can be extended to targeting intracellular proteins, disease markers, etc.

### BRIEF DESCRIPTION OF THE DRAWINGS

FIG. 1 shows silica beads coated with an analyte capturing reagent placed in a tube with an inlet and outlet and a filter.

FIG. 2 shows a sensitive fluorescence detection method that can detect low concentrations of intracellular fluorescent tagged cells in solution.

### DETAILED DESCRIPTION OF EXEMPLARY EMBODIMENTS

Disclosed herein are methods to perform intracellular fluorescent assay in solution (IFAIS) for various applications.

As used in the specification and claims, the singular form “a”, “an” and “the” include plural references, unless the context clearly dictates otherwise. For example, the term “a capillary” may include a plurality of capillaries coupled together.

The method of the invention enables the rapid and efficient detection and quantification of target cells in a test sample. The test sample can be a biological liquid such as blood. The test sample is preferably in the form of a solution or suspension of the target cell that can be introduced directly into the fluorometer. It is not necessary to deposit the test sample on glass slides or dry the sample. In one embodiment, the target cells are proteins or disease markers.

In one embodiment of the invention, the test sample is contacted with a solid support to collect and concentrate the target cells. The solid support can be a filter or beads having a coating of an analyte having a binding affinity for the target cells. The beads can be magnetic beads, solid glass beads or hollow glass beads.

The target cells are then treated with a fluorescent dye to tag the cells. The tagged cells are dispersed into solution and introduced to a fluorometer to detect and quantify the tagged cells. The cells can be lysed or whole. In one embodiment, the cells can be removed from the magnetic or glass beads and dispersed in solution.

Various fluorescent dyes can be used in the method of the present invention. The fluorescent dye is selected depending on the target cells, and the concentration of the target cells in the solution. It has been found that fluorescent dyes that fluoresce in the red and far infrared range are particularly suitable for certain applications. The fluorescent dyes that the fluorescent emissions do not significantly overlap the Raman emission of water provide good sensitivity in the fluorometer. Raman emission of water introduces high background. For example, TexasRed (sulforhodamine 101 acid chloride), absorbing at 589 nm and emitting at 615 nm, and Cy™-5 and similar dyes, absorbing at 635 nm and emitting at 670 nm, are suitable. Additional examples of suitable dyes are DyLight series of dyes 638/658, 654/673, 692/712 excitation/emission wavelength in nm, and Alexa Fluor series of dyes 590/617, 612/626, 632/647, 633/647, 650/665, 663/690 excitation/emission wavelength in nm. Other long wavelength fluorescent dyes can also be used. In some applications, the short wavelength fluorescent dyes are suitable but may not provide the sensitivity to exhibit rapid detection.

The following description facilitates a thorough understanding of the invention for purposes of explanation, but not limitation. The specific details set forth particular embodiments of the IFAIS. The invention can, however, be practiced in other embodiments that depart from these specific details.

I. Detection of Pathogens in Blood

There are various options to concentrate and isolate micro-organisms from complex matrices, such as blood, for FISH or PNA FISH assays. The methods can vary, depending on the type of sample and the type of cells. The method of concentration or analyte enrichment can utilize magnetic beads, solid silica (glass) beads, hollow silica spheres, micro- and nanofilters, and combinations thereof. The magnetic or silica beads define a solid support for capturing the target cells. The beads have a coating or surface layer having an analyte with a binding affinity for the target cells. A few examples will be given for the detection of pathogens in blood.

I.A. IFAIS Using FISH or PNA FISH and Magnetic Bead Enrichment

1. Magnetic Bead Capture

Magnetic beads are prepared, such that the surface is conjugated with a pathogen capture reagent that can specifically capture the pathogen(s) of interest.

Magnetic beads can be directly added to the blood to capture the bacterial cells.

Alternatively, an appropriate amount of the magnetic beads is added into a blood culture bottle. Patient blood sample is inoculated into the blood culture medium. The bottle is incubated at 37° C. with motion to provide mixing of the magnetic beads in the blood culture bottle. The magnetic beads will capture the pathogen while it is multiplying, producing copious amount of rRNA in the cells for FISH or PNA FISH. After a predetermined length of time, a portion of or all of the sample from the bottle can be taken out for testing. The magnetic beads with the captured cells are separated from the solution and collected by a magnet to provide concentration. The magnetic beads are washed with PBS buffer to remove the remaining culture medium. In one embodiment, the target cells can be retained on the magnetic beads or removed from the beads using standard procedures.

2. Permeabilizing the Cell Membrane

The cell membranes are permeabilized using low concentration of ethanol to allow penetration of the PNA FISH probes. An example of the treatment regimen is to use ≦10% ethanol for the required time, typically >10 min. These conditions can vary depending on the cell. The solution containing ethanol is removed and the beads allowed to dry.

The cell membranes can also be permeabilized using 80-96% ethanol for 5-10 min or longer to allow penetration of the probes. The high concentration of ethanol will denature the antibody and release the cells from the beads. The beads are isolated by a magnet, and the solution without beads collected and placed into a filter tube with small pore size <0.2 μm to separate the bacteria from the solution. The solution containing ethanol passes through the filter, leaving the cells on the filter.

3. FISH or PNA FISH

Add FISH or PNA FISH reagent to the pathogen. Suitable reagents can include PNA FISH reagent targeting rRNA of a specific bacteria, or rRNAs of a group of bacteria, conjugated with fluorescent dyes that have emission efficiency and the fluorescent emission and Raman emission do not overlap. Cover the sample to prevent evaporation and incubate for the time and temperature appropriate for the reagent. Wash the cells in wash solution at the appropriate temperature.

4. Florescence Detection

The fluorescent signal of the sample may be measured in one of the following manners:

a. The sample can be the whole cells attached to the magnetic beads,

b. The sample can be the whole cells detached from the magnetic beads,

c. The cells can be lysed with the magnetic beads still in the solution, or

d. The cells can be lysed with the magnetic beads removed from the solution.

To read the sample in a fluorometer, collect the sample of cells or lysate in solution and place it into the sample holder of the appropriate fluorescence detection platform.

A variety of fluorometers can be used. One common platform is the fluorescent plate reader. The total number of cells preferably in each well of the plate reader are to be greater than 10⁵.

One sensitive fluorometer that is suitable for small samples and low analyte concentrations, which is not based on the plate reader format, is based on integrating waveguide technology (IWT). For the IWT, the emission reagents are in solution inside a waveguide. The waveguide can be a clear glass capillary cuvette. An example is Roche Light Cycler PCR tube. The solution and the capillary tube together act as a waveguide. A light source appropriate to the fluorescent dye illuminates the capillary waveguide containing the solution from a direction perpendicular to the length of the waveguide. The emitted fluorescent signal is efficiently gathered by the waveguide and propagates to the ends of the waveguide. The fluorescent signal exits from the end of the waveguide. One implementation of the IWT is a spectrofluorometer available under the trade name Signalyte™-II. The total number of cells preferably in each 35 μm of the Roche Light Cycler PCR tube is approximately 10 or more using Signalyte™-II. This can be concentrated from much larger volume of sample, such as 1 ml, but the sample size could be larger.

Experiments were performed to show detection of E. coli O157 by PNA FISH in solution using reagents from AdvanDx. For comparison, the same serial dilutions of E. coli hybridized with Texas Red were tested on a BMG FLUOstar Omega fluorescent plate reader (200 μl samples in the 96 well plate) and Signalyte™-II (40 μl samples in Roche Light Cycler PCR tubes). In these experiments, Signalyte™-II was more than five orders of magnitude more sensitive than the 96 well plate reader.

I.B. IFAIS Using FISH or PNA FISH and Solid Silica Beads or Hollow Silica Microspheres for Enrichment

1. Solid Silica Bead or Hollow Silica Microsphere Capture

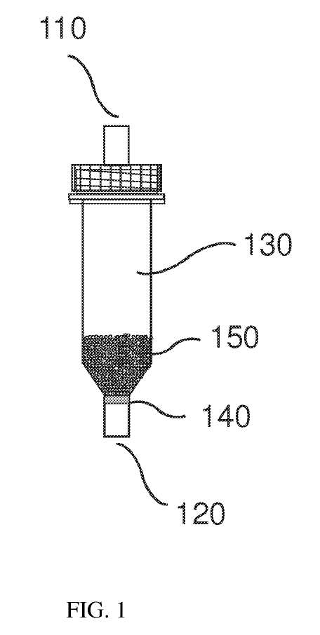

Solid silica (glass) beads or hollow silica microspheres coated with pathogen capture reagent, such as antibody against for a specific pathogen, can be used to enrich the concentration of the pathogens in the sample test solution. Use of silica beads or microspheres is particularly suited for samples larger than 1 ml size. An example is shown in FIG. 1. Beads or microspheres 120 can be placed in a tube 130 that can be connected to an input 110 and an output 120. The beads 150 are prevented from flowing out of the tube by a filter 140. The beads or microspheres are larger than the pore size of the filter. The sample containing the target cells or pathogens is passed through the bed of beads. The sample can be passed through the beads more than once. The pathogens are captured on the silica surface of the beads. The silica beads and microspheres are washed with PBS buffer to remove remaining culture medium while the captured pathogen remains on the surface of the beads.

2. Permeabilizing the Cell Membrane

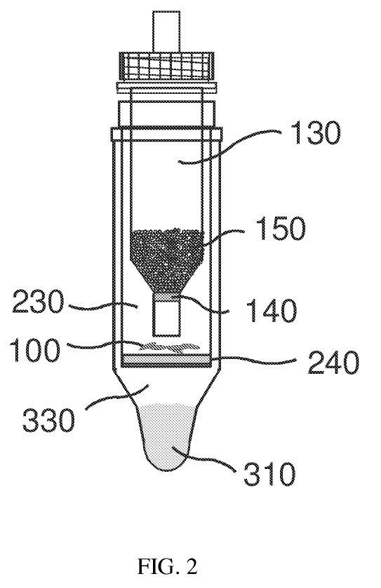

The cell membranes can be permeabilized using 80-96% ethanol for 5-10 min or longer to allow penetration of fluorescent probes. The high concentration of ethanol will denature the antibody and release the cells from the beads. The tube containing the beads 130 is placed inside a filter tube 230, which is placed inside a collection tube 330. Ethanol is collected into the collection tube 330 and the cells 100 collected in a filter tube 230 with small pore size <0.2 μm to separate the cells from the solution. This is done by centrifuge using the setup shown in FIG. 2. The solution containing ethanol passes through the filter 240, and cells 100 are retained in the filter tube 230.

3. FISH or PNA FISH

Add FISH or PNA FISH reagent to the sample 100 in the filter tube 230. Cover the tube to prevent evaporation and incubate for the time and temperature appropriate for the reagent. Wash the cells in wash solution at the appropriate temperature.

4. Florescence Detection

The cells can be collected or lysed and the solution eluted. The solution is read in a fluorometer, and placed into the sample holder of the appropriate fluorescence detection platform.

I.C. IFAIS Using FISH or PNA FISH and Using Hollow Silica Microsphere for Enrichment

Hollow silica microspheres float in water. An alternative method to capture the cells is to place silica microspheres and the sample in a container. Examples of container are a culture tube or centrifuge tube. The tube is placed in a rotator to allow end to end mixing. The cells are captured on the beads. The silica microspheres float to the top in a short time and are collected by pipette and washed with PBS buffer to remove the culture medium matrix. The steps to permeate the cells, incubating with FISH or PNA Fish, and reading the eluted solution with the cells or lysed cells are the same as described in Section I.B.

Variations for Pathogen Detection in Blood for Examples I.A, I.B and I.C

Silica beads, hollow silica microspheres, magnetic beads and other capture products can be conjugated with a cell recognition reagent. They can be used to concentrate cells in the same manner as magnetic beads.

The beads can be conjugated with a recognition reagent, such as antibodies, aptamers, proteins, carbohydrates or chemicals, such as polylysine and polymyxin-B that recognizes cell surfaces of interest.

The test sample can be whole blood, components of blood, blood culture inoculated with patient blood or urine.

The number of pathogens to be detected at one time can be more than one. For example, if the fluorescent instrument provides four excitation and emission wavelengths, then four different pathogens can be tested each at a different excitation/emission wavelength.

More than one property or component of a given cell can be detected at one time using fluorescent dyes with different emission wavelengths.

I.D. IFAIS Using FISH or PNA FISH and Filter Set Concentration and Purification of Bacteria in Blood

1. Enriching Bacterial Cells by Filters

When there is a large variety of cells or an unknown variety of cells to be concentrated, the use of magnetic beads can become impractical. An example of such a need is the detection of unidentified bacteria in blood. Bacteria are typically smaller in size than blood cells. For example, E. coli is a short rod like cell 0.5 μm in cross section×1 μm long. Blood cells are larger than 2 μm. The use of size specific filters and combinations of filters may be the economical choice.

An example is the combination of large pore filters to remove blood cells and small pore filters to retain the bacteria. The sample can be whole blood, components of blood, blood culture inoculated with patient blood, or diluted blood.

First, the sample passes through a filter with pores large enough for bacteria to pass but small enough to retain the blood cells. The pores can be 2-3 microns in diameter. When there are a lot of blood cells and clogging could be a problem, the hollow fiber type of filter should be used, and the sample should be dilute to allow flow. Place the eluted sample into second filter tube with pore size much smaller than the cell, such as <0.2 μm. The bacterial cells are retained in this second filter. This is followed by washing.

2. Permeabilizing the Cell Membrane

The cell membranes can be permeabilized using 80-96% ethanol for 5-10 min or longer to allow penetration of the fluorescent probes. After incubation, ethanol is removed from the filter.

3. FISH or PNA FISH

Add FISH or PNA FISH reagents to the pathogen on the filter. Incubate for the time and temperature appropriate for the reagent. Wash the cells in wash solution at the appropriate temperature.

4. Florescence Detection

Whole cells can be removed from the filter and tested. Alternatively, the cells can be lysed and the solution eluted. The solution is read in a sensitive fluorometer.

The number of pathogens to be detected at one time can be more than one. For example, if the fluorescent instrument provides four excitation emission wavelengths, then four different pathogens can be tested, each at a different excitation/emission wavelength.

II. Detection of Cells in Body Fluids.

In addition to blood cells, other cells that can be found in blood are circulating tumor cells, fetal cells, epithelial cells, etc,

Body fluids can be peripheral blood, spinal fluid, saliva, urine, tears, mucus, etc.

II.A IFAIS Using Magnetic Beads, Silica Beads, or Silica Microspheres for Enrichment of Circulating Tumor Cells in Blood

The procedure is similar to enrichment of pathogenic organisms in blood, except that the appropriate reagents for the analyte of interest coated onto the magnetic beads, silica beads or silica microspheres, and the intracellular fluorescent reagents.

II.B IFAIS Using Precision Microfilter for Concentration of Circulating Tumor Cells in Blood

1. Enrichment of Circulating Tumor Cells Using Precision Pore Filters

Circulating tumor cells in blood are typically from about 12 microns to greater than 35 microns, larger than most red and white blood cells. Pores 7-8 μm in diameter can retain most circulating tumor cells and eliminate nearly all the blood cells. Precision pore filters can be fabricated by track etch method, UV, x-ray and energetic neutral atoms lithography fabrication methods.

The sample first passes through a filter with pores large enough to pass most of the blood cells, but small enough to retain circulating tumor cells. The pore dimensions should be precise. The pore size 7-8 μm in diameter is used most often in research. Breast cancer cells are typically 30 μm in diameter, so that larger pores can be used for breast cancer cells. The circulating tumor cells are retained in the filter. This is followed by washing.

2. Permeabilizing the Cell Membrane

The cell membranes are permeabilized using low concentration of ethanol to allow penetration of fluorescent probes. An example of this regimen is to use ≦10% ethanol for the required time, typically >10 min. These conditions can vary depending on the cell. The solution containing ethanol is removed, and the filter allowed to dry.

3. Biomarker

Add fluorescent biomarker reagent to the CTCs on the filter. Incubate for the time and temperature appropriate for the reagent. Wash the cells in wash solution at the appropriate temperature.

4. Florescence Detection

The cells can be tested as whole or lysed. The solution containing the cells is placed into the sample holder and read using a sensitive fluorescence detection platform.

The number of types of cells and the number of markers to be detected at one time can be more than one, depending on the number of excitation/emission wavelengths provided by the fluorescent detection instrument.

### EXAMPLES OF APPLICATIONS

A few examples of applications are described below. The potential applications are not limited by the following examples. Because of the differences of the matrix and cells, each assay will have to be modified accordingly.

### Example 1

### Test Bacteria in Blood

Every year, 350,000 patients acquire bloodstream infections in the U.S. resulting in more than 90,000 deaths and significant costs to the healthcare system. Conventional diagnostic methods for bacteremia, consisting of blood culture (>8 hours) followed by subculture on agars, can take several days. Before obtaining the diagnostic test result, doctors might administer broad spectrum antibiotics, which can be expensive, toxic and even unnecessarily contribute to antibiotic resistance. Ineffective or incorrect treatment leads to increased mortality, morbidity, length of stay, and overall hospital cost.

### Example 2

### Testing for Bacteria in Urine for Urinary Infection

### Example 3

### Prognostic Test for Chronic Lymphocytic Leukemia (CLL) Using ZAP-70 as Marker in B Cells

B cells are enriched using magnetic beads, silica beads or hollow silica microspheres coated with antibody against CD19. ZAP-70 inside whole B cells from CLL patients are tagged by fluorescent labeled antibodies against ZAP-70. These cells are typically read by flow cytometry. These cells can be read in solution using a sensitive fluorometer, such as Signalyte™-II.

### Example 4

### Testing Cervical Cancer Cells by FISH or PNA Fish in Solution

### Example 5

### Testing for Tumor Cells in Body Fluids

Circulating tumor cells in blood and bladder tumor cells in urine can be enriched using magnetic beads, silica beads, silica microspheres, or precision filters. Intracellular fluorescent assay in solution (IFAIS) to one or more disease markers of the captured cells can provide quantitative and disease information using a sensitive fluorometer.

### Example 6

### Testing of Bacteria in Food Using FISH or PNA Fish Reagents

Bacteria in food can cause serious diseases. Foods are required to be tested for a variety of bacteria as regulated by FDA and USDA. Bacteria in food are tested by plating, immunoassay and real time PCR. Use of FISH or PNA FISH is an alternative method for molecular detection.

### Example 7

### Testing of Bacteria in Water and Environment Using FISH or PNA FISH Reagents

While the invention has been shown and described with reference to certain exemplary embodiments thereof, it will be understood by those skilled in the art that various changes in form and details may be made therein without departing from the spirit and scope of the invention as defined herein.

What is claimed is:

1. A method of detecting pathogens in a sample containing body fluids and cells from tissue samples, the method comprising:
isolating target cells from the sample containing the body fluids or cells form the tissue sample;
tagging the target cells with a fluorescent reagent;
isolating the target cells with the fluorescent reagent and forming a solution thereof; and
introducing the solution of the target cells to a fluorometer and fluorescing the target cells in solution to detect the pathogens.

2. The method of claim 1, wherein the fluorometer is by integrating waveguide technology (IWT).

3. The method of claim 1, wherein the target cells are proteins or disease markers.

4. The method of claim 1, wherein the fluorescent agent is a FISH or a PNA FISH reagent.

5. The method of claim 1, wherein the target cells are isolated from the body fluid or tissue sample by contacting the sample with a solid support having a binding affinity for the target cells and recovering the target cells from the solid support.

6. The method of claim 5, wherein the solid support comprises magnetic beads or glass beads having a binding affinity for the target cells, said method further comprising
contacting the target cells with the magnetic beads or glass beads and adhering the target cells to the magnetic beads or glass beads,
recovering the magnetic beads or glass beads from the sample, and
recovering the target cells from the magnetic beads or glass beads.

7. The method of claim 5, wherein the solid support is a filter and said method further comprises
passing the sample through the filter having a pore size sufficient to recover the target cells and recovering the target cells from the filter.

8. The method of claim 5, wherein the solid support comprises hollow glass beads having a binding affinity for the target cells, and said method further comprises
contacting said target cells with said hollow glass beads for a time sufficient to attach the target cells to the hollow glass beads,
recovering the hollow glass beads from the sample, and
recovering the target cells from the sample.

9. The method of claim 6, further comprising
dispersing the magnetic beads or glass beads having the target cells adhered thereto in solution, and subjected to said fluorescing in the fluorometer in solution.

10. The method of claim 9, wherein the targets cells are lysed prior to fluorescing.

11. The method of claim 9, wherein the target cells are whole.

12. The method of claim 6, further comprising the step of
permeating the cell membrane of the target cells on the magnetic beads or glass beads,
applying FISH or PNA FISH reagents, conjugated with fluorescent dyes, and
suspending the recovered target cells in solution.

13. The method of claim 12, further comprising lysing the cells prior to loading the sample into sample holder for testing.

14. The method of claim 1, wherein the cells are selected from the group consisting of pathogens, micro-organisms, tumor cells, fetal cells and epithelial cells.

15. The method of claim 1, wherein the fluorescing agent is a fluorescing dye.

16. The method of claim 15, wherein the fluorescent dyes have the property that the emission wavelengths do not significantly overlap the Raman emission wavelength of the water.

17. The method of claim 1, wherein the body fluid is selected from the group consisting of blood, spinal fluid, saliva, urine, tears and mucus.

18. The method of claim 1 wherein the fluorometer is a plate reader.

19. A method of detecting target cells in a test sample, the method comprising the steps of
providing a test sample solution containing the target cells and contacting the test sample solution, a solid support having a binding affinity for the target cells to bone the target cells to the solid support,
recovering the solid support from the test sample solution,
permeating the cell membrane,
tagging the target cells on the solid support with a fluorescent dye,
separating the target cells from the solid support and forming a solution, and
introducing the solution containing the target cells into a sample holder for a fluorometer that can excite the fluorescence of the fluorescent dye and detect the emission of the dye to determine the presence of the target cells.

20. The method of claim 19, wherein said target cells are pathogens.

21. The method of claim 19, wherein said target cells are E coli.

22. The method of claim 21, wherein the method further comprises
lysing the target cells in solution before introducing the solution into the fluorometer.

23. The method of claim 22, wherein the dye is TexasRed.
